(19) United States
(12) Patent Application Publication
(10) Pub. No.: US 2008/0077445 A1
(43) Pub. Date: Mar. 27, 2008

(54) METHOD OF HEALTHCARE DELIVERY WITH VALUE STREAM MAPPING

(75) Inventor: Robert S. Mecklenburg, Seattle, WA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: VIRGINIA MASON MEDICAL CENTER, Seattle, WA (US)

(21) Appl. No.: 11/534,762

(22) Filed: Sep. 25, 2006

(51) Int. Cl. G06F 19/00; A61B 5/00

(52) U.S. Cl. 705/3; 600/300

(57) ABSTRACT

The invention relates to systems and methods for the delivery of healthcare. The inventive methods are based on the identification of and the partial or complete removal of waste from the value stream of healthcare delivery for a range of clinical services. Evidence-based medicine and/or cost accounting methodologies may be used in accordance with various embodiments of the present invention. The inventive methods bring together a number of parties (e.g., payor, provider and employer) to achieve improvement goals as well as optimization of healthcare delivery from all perspectives, as opposed to doing so at the expense of one or more parties.

### FIELD OF INVENTION

The invention relates to the delivery of healthcare services. Specifically, the invention relates to various processes for improving the quality and efficiency of healthcare delivery while reducing costs.

### BACKGROUND OF THE INVENTION

The Toyota Production System (“TPS”) is a framework and management philosophy organizing the manufacturing facilities at Toyota as well as the interaction of these facilities with Toyota's customers. The main goals of TPS are to improve quality and efficiency through the elimination of waste, which, in the application of TPS at Toyota, includes defects, overproduction, transportation, waiting, inventory, motion and processing. By employing TPS, Toyota has been able to greatly reduce cost and inventory, enabling it to become one of the largest companies in the world. TPS is an example of the kaizen (from Japanese, meaning “continuous improvement”) approach to productivity improvement. Due to the success of this production philosophy, many of these methods have been copied by other manufacturing companies. See, e.g., TAIICHI OHNO, TOYOTA PRODUCTION SYSTEM (1995), YASUHIRO MONDEN, TOYOTA PRODUCTION SYSTEM (3rd ed. 1998), and JEFFREY LIKER, THE TOYOTA WAY (2003), each of which is incorporated herein by reference as though fully set forth.

Most companies that implement TPS use a typical business model, in which the customer and the company are clearly defined entities. Typically, the company's goal is to learn what the customer wants and is willing to pay for; make it and sell it to them; pay its expenses; and treat the remainder as profit. The company sets its own pricing and therefore exercises a certain amount of control over its profits (or lack thereof). Failure criteria tend to be clear, and falling quality can have relatively quick and painful consequences for the company—if sales fall, complaints rise, profits and share price go down and the company suffers directly. Using TPS, the company can reduce waste to increase profits and boost quality, according to customer needs.

In healthcare, the situation is far more complex. To begin with, there are at least four entities that are typically involved; not just two, as with the company/customer dynamic that is typical to the consumer products arena. These four entities may include: (1) the payor (i.e., the entity that pays the healthcare provider for the product), (2) the employer (i.e., the entity that pays the payor to pay the provider for the product), (3) the provider (i.e., the entity that gets paid for the product), and (4) the patient (i.e., the individual who directly receives the product). Moreover, the complexity of the clinical and financial relationships among these parties, in addition to other external forces, such as complicated state and federal regulatory regimes, renders the implementation of TPS in the healthcare setting a challenging feat. It is believed that the basic TPS model that is used in the consumer products arena cannot merely be used in the healthcare setting absent significant modifications and enhancements.

The foregoing examples of the related art and limitations related therewith are intended to be illustrative and not exclusive. Other limitations of the related art will become apparent to those of skill in the art upon a reading of the specification and a study of the drawings. All references cited herein are incorporated by reference as if fully set forth.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In one embodiment, the invention includes a method for delivery of a healthcare service, comprising: gathering data relating to the delivery of the healthcare service; manipulating the data to place it in a format suitable for a current state value stream map; creating the current state value stream map; identifying waste in the current state value stream map; creating a future state value stream map including at least one proposed change by removing at least a portion of said waste; and implementing the healthcare service based on the future state value stream map. The method may further comprise testing and/or simulating the at least one proposed change prior to implementing the healthcare service based on the future state value stream map. The current state value stream map may comprise a current map item selected from cycles of work, wait times between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, patient satisfaction scores associated with the current state value stream, work days lost associated with the current state value stream, and combinations thereof. Creating the current state value stream map may further comprise creating the map for one episode treatment group. Creating a future state value stream map may further comprise utilizing an evidence-based medicine approach to propose the proposed change. Waste may be selected from a defect, an overproduction, a transportation, a waiting, an inventory, a motion, a processing, an inefficiency, a non-value added element of said current state value stream map, a lengthy delay between cycles, an expensive treatment and/or tests determined to be redundant and/or that adds little or no value to the final product, a provider visit that is unnecessary, and combinations thereof. The future state value stream map may depict a provision of the healthcare service with substantially all of the waste having been eliminated. The future state value stream map may comprise a future map item selected from a cycle of work, a wait time between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, a new measure that results from the implementation of the future state value stream map, a reduced lead time, a reduced number of days of work lost, an increase in patient satisfaction metrics, an increased percentage of value-added time in said lead time, a non-value-added activity eliminated, and combinations thereof. Creating the future state value stream map may further comprise conducting a negotiation between a payor and a provider or between the payor, the provider and an employer. Implementing the healthcare service based on the future state value stream map may further comprise an implementation activity selected from changing a standard order set, using an algorithm, creating a form, utilizing online test ordering forms with decision rules built-in, training staff and/or physicians to learn and implement the healthcare service based on the future state value stream map, creating printed material to facilitate the training, and combinations thereof. A payor may pay a provider less per episode of care as a result of implementing the healthcare service based on the future state value stream map. A payor may pay a provider more per episode of care as a result of said implementing said healthcare service based on said future state value stream map and an outcome is of greater quality and/or a patient satisfaction is higher and/or a number of work days lost is lower. In an embodiment, the method further comprises repeating the method to further improve the delivery of the healthcare services.

In another embodiment, the present invention includes a future state value stream map for the delivery of a healthcare service, produced by the process of: gathering data relating to the delivery of the healthcare service; manipulating the data to place it in a format suitable for a current state value stream map; creating the current state value stream map; identifying waste in the current state value stream map; and creating the future state value stream map including at least one proposed change, by removing at least a portion of the waste. The process may further comprise utilizing an evidence-based medicine approach to propose the proposed change. Waste may be selected from a defect, an overproduction, a transportation, a waiting, an inventory, a motion, a processing, an inefficiency, a non-value added element of the current state value stream map, a lengthy delay between cycles, an expensive treatment and/or tests determined to be redundant and/or that adds little or no value to the final product, a provider visit that is unnecessary, and combinations thereof. The future state value stream map may depict a provision of the healthcare service with substantially all of the waste having been eliminated. The future state value stream map may further comprise a future map item selected from a cycle of work, a wait time between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, a new measure that results from the implementation of the future state value stream map, a reduced lead time, an increased percentage of value-added time in said lead time, a non-value-added activity eliminated, and combinations thereof. The process may further comprise conducting a negotiation between a payor and a provider or between the payor, the provider and an employer.

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1 depicts a flow chart of a process for improved delivery of healthcare, in accordance with an embodiment of the present invention.

FIG. 2 depicts a current state value stream map in accordance with an embodiment of the present invention.

FIG. 3 depicts a future state value stream map in accordance with an embodiment of the present invention.

### DETAILED DESCRIPTION OF THE INVENTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

The present invention is based on the application of TPS principles, in general, in the healthcare context. It uniquely brings together a team of the payor, provider and employer to achieve improvement goals, thus ensuring agreed-upon common definitions of “improvements,” “quality,” and “value,” as well as optimization of healthcare delivery from all perspectives, as opposed to doing so at the expense of one or more parties. The invention thus provides a methodology for aligning reimbursement with value.

Various embodiments of the present invention integrate the elements of the clinical flow, provider costs and accounting principles, reimbursement claims data, evidence-based medicine, and the like, and then apply principles of value stream mapping, waste reduction and other elements of TPS to the healthcare setting to define, identify and compare reimbursement with value. Thereby, various embodiments of the present invention incorporate the needs of multiple parties, including payor, employer, provider and patient. The inventive systems and methods provide a framework for aligning incentives of these parties so that providers may be rewarded, not penalized, financially, for, among other things, reducing costs, improving outcomes, improving efficiency, reducing barriers to flow of value and the like. By application of the inventive system and method, the parties' incentives are aligned such that all involved stakeholders can benefit.

The development of various embodiments of the present invention—its tools, methods and systems—has required significant modification from the TPS. This is based on the many qualities that differentiate the manufacturing industry from the delivery of healthcare to patients. TPS was created and is chiefly applied in a manufacturing context, while healthcare is a service context. In addition, manufacturing in the automotive industry is quite different in many respects from services offered in the healthcare industry. As noted above, for example, there are more parties involved in the delivery of healthcare services (e.g., payor, employer, provider and patient) than in the manufacturing context (e.g., company and customer). In fact, the tools and methods that pertain predominantly to automotive parts and other physical items and flows in TPS are not all directly applicable in the same manner to delivery of healthcare services. Such approaches require significant modification and re-interpretation to be relevant to patient needs and to the flows of patients, information, equipment and supplies, providers and process improvement in the healthcare setting. Simply by way of example, in sharp contrast to a conventional manufacturing context, in the healthcare setting clinical decisions are made with regard to specific patient conditions rather than determinations as to how to operate a specific manufacturing machine.

As will be readily appreciated by those of skill in the art, the inventive systems and methods can be applied to any malady/condition and corresponding clinical/medical service offered in the healthcare setting.

In one embodiment of the present invention, value stream mapping may be utilized. As used herein, a “value stream map” is defined as an illustration of an entire lead time for a service or product. A value stream map may illustrate all cycles of work as well as the wait times in between the cycles of work to produce one complete episode of care from initiation of demand to care completed. As used herein, a “cycle of work” is defined as one activity completed within a lead time and may involve multiple operators. A value stream map may include calculations of time for each cycle of work, work in process between cycles, information and/or material flows, improvement (kaizen) opportunity flags identifying defects and wastes in the process, and/or calculation of the percentage of time that is value-added. The value stream map may also include information regarding costs, quality, patient satisfaction and/or employer key metrics (e.g., work days lost). The “current state” referenced in a value stream map is a baseline, depicting how a product/service is conventionally delivered (i.e., the prior art). The “future state” referenced in a value stream map depicts how the product/service may be delivered with a significant reduction in the amount of waste (in some embodiments, without any waste) in the value stream, as a result of the application of the techniques of the present invention.

Thus, value stream mapping may be used in accordance with various embodiments of the present invention to map and evaluate current flow. In one embodiment, a value stream map may be created for one instance of an episode treatment group (“ETG,” or diagnosis) from start (i.e., patient's first contact with the provider) to finish (i.e., patient has completed all treatments and visits). In another embodiment of the present invention, provider costs may be compared with payor reimbursements for every service in a value stream map. In a still further embodiment of the present invention, evidence-based medicine may be used to guide improvements in healthcare delivery. This may be in addition to one or more of the standard seven “wastes” used in TPS (i.e., defects, overproduction, transportation, waiting, inventory, motion and processing). While not wishing to be bound by any particular theory, it is believed that the integration of the process flow, illustrated by the value stream map, the cost accounting, the claims data and the evidence-based medicine are unique features of the instant invention.

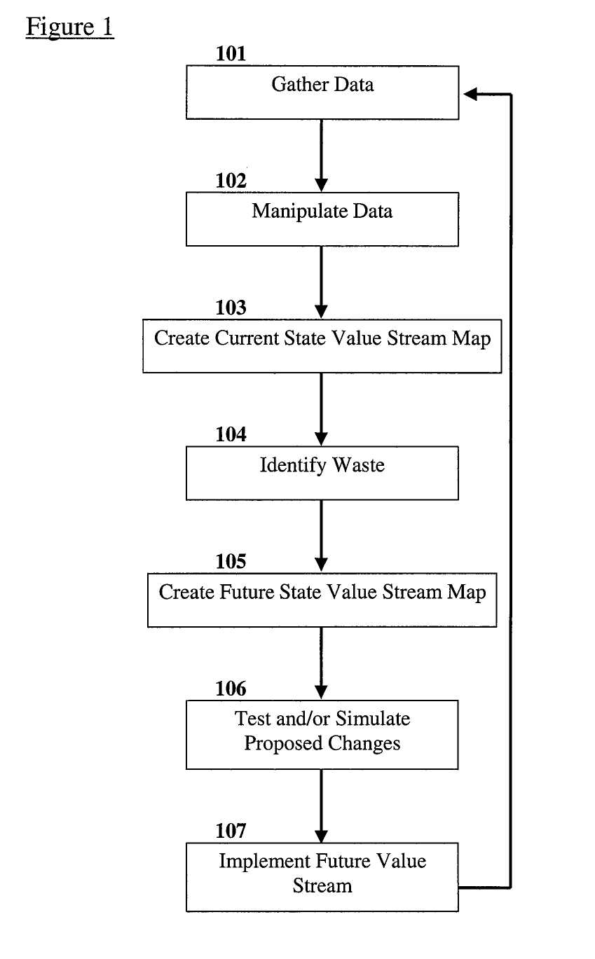

As illustrated in FIG. 1, a value stream map may be created by first gathering data 101. A wide array of data may be gathered in accordance with alternate embodiments of the present invention, as will be readily understood by those of skill in the art. Depending upon the particular application of the present invention, the type, quantity and format of data may be modified.

In one embodiment, a ranking of ETGs by the cost of each ETG to the payor (e.g., most expensive to least) may be collected, and thereafter used to prioritize improvement work. Generally speaking, payors are more likely to consider comparatively expensive ETGs important items for cost improvement as opposed to comparatively less expensive ETGs. However, as one of skill in the art will readily recognize, there are a number of other considerations that might impact the manner in which ETGs are prioritized, such as, by way of example, the volume of use of particular services relative to one another.

In another embodiment, documentation of flow of patients through one complete lead time to receive a clinical service and/or product may be collected. This documentation may include a number of items; for example, the total lead time, descriptions and times of every cycle of work performed by each operator (e.g., clinical and non-clinical staff, providers, provider suppliers and vendors, transporters, ancillary services, and the like), times of all waits in between cycles, the percentage of produced services and/or products containing defects (wherein defects are defined specifically with respect to subject matter of value stream), “takt time” for the product and/or service (i.e., the pace of customer demand for the product and/or service; this may be calculated as hours available to do work divided by total demand during those hours), work in process of the product and/or service (i.e., the number of products, services, or patients in the value stream that are not completed), the inventory of supplies used in delivering the product and/or service, and/or the value-added time versus non-value-added time. As one of skill in the art will readily appreciate, still further documentation may be collected to supplement any or all of the foregoing examples of documentation and information.

In another embodiment, financial data may be collected. This may include, for instance, the total cost (distinguished as direct, indirect, fixed, etc.) to the provider for providing one complete ETG; such costs may be further categorized by each service or treatment within the value stream map of the ETG. It may also include the total reimbursement to the provider (i.e., cost to payor) of providing one complete episode of care, which may be further categorized by each service or treatment within the value stream map of the ETG. Again, as one of skill in the art will readily appreciate, still further data may be collected to supplement any or all of the foregoing examples of financial data.

The aforementioned data may be gathered through any number of methodical processes. The process selected for gathering any or all of this data, or indeed still further data, may be dependent upon not only the type of data being collected, but also the nature of the systems used within a particular institution to store such data, the number of personnel and/or computerized systems available to aid in the exercise of gathering data, and any number of additional factors that will be readily apparent to those of skill in the art.

In one embodiment of the present invention, the value stream map data may be gathered by direct observation in the place where the work is actually performed. An observer may observe each activity, from beginning to end of the lead time of the product/service, and may document each such activity, from the patient's perspective. Movements of operators, patients, supplies, equipment, medications, and the like may be sketched using tools specific to this task. An observer may document individual tasks and steps as well as the time (e.g., minutes/seconds) required to perform them. The observer may also tally the work in process in the place where the work is actually performed, assess the presence of “push” or “pull” in workflow, and note and tally specific defects in the process. The resulting data may then be transferred (e.g., in a standardized form) to a value stream map, whereby a current state value stream map may be created.

Financial data may also be gathered. The total costs to the provider may be obtained from the provider institution's costing system software, and may include, for instance, staff and physician payroll data, overhead/allocated costs, supply costs, and the like. Total reimbursement data may be collected based upon the payor's claims data and rates for individual services (e.g., based on CPT codes).

Various personnel may be involved in gathering any or all of the aforementioned data in connection with alternate embodiments of the present invention. This may include, by way of example, the provider's staff, employees and others who perform the actual work described in the value stream map; members of a team created to gather such data; the provider's finance staff; the payor, its staff, employees and others; and the patient's employer, its staff, employees and others.

As illustrated in FIG. 1, once collected, the aforementioned data may be manipulated 102 in any number of ways in connection with alternate embodiments of the present invention. In one embodiment, where there are multiple observations of a single cycle or multiple measures of a lead time, the most representative value is selected for use in the current state value stream map. In various embodiments, the most representative value may be the mode or the median of the embodiments for which data is collected. With respect to financial data, where only aggregate and/or batched dollar data is available, these may be allocated and/or averaged.

As illustrated in FIG. 1, the output of value stream mapping may be a complete, current state value stream map 103. Opportunities for waste reduction may be identified in the current state value stream map 104. The data collected may also be displayed in the current state value stream map.

As used herein, “waste” refers to any non-value added elements of the value stream map, and may include, but is in no way limited to, lengthy delays between cycles, expensive treatments and/or tests that are determined to be redundant and/or that add little or no value to the final product, provider visits that are unnecessary, and the like. Inefficiencies and/or opportunities may be identified in a number of ways, either alone or in combination, such as by identifying one or more of the seven wastes referenced in TPS; by using evidence-based medicine to assess every activity occurring in the current state value stream map and determining whether each is necessary/desirable or redundant/superfluous; by noting where costs are exceeding reimbursements (e.g., per cycle and aggregated across an entire value stream); and the like. Still further mechanisms by which to identify inefficiencies and/or opportunities may be apparent to those of skill in the art; in particular, this may vary in connection with the type of healthcare service being examined.

A future state value stream map may be created 105 based on the removal of waste, inefficiencies and the like from the current state value stream. The future state value stream map may depict the provision of the care to the patient with these identified wastes, inefficiencies and the like eliminated. It may also depict the new measures that may result (e.g., reduced lead time, increased percentage of value-added time in the lead time, non-value-added activity eliminated, etc.) if the future state value stream is implemented. The future state value stream map may thus be used to guide the delivery of healthcare services or for other purposes that will be readily apparent to those of skill in the art.

For each ETG being improved, a team, including representatives from the provider, payor, and employer, may convene on a regular basis to assess the current state value stream map and decide how to improve it based on the opportunities identified. The team may test and/or simulate all proposed changes 106, according to the rules of kaizen, PDSA (i.e., plan, do, study, act) and/or any other suitable approach. In one embodiment of the present invention, no change can be implemented without having been tested. If tests, visits, treatments or other cycles are to be eliminated, negotiation may need to take place between payor and provider. As wasteful services are removed, costs may be eliminated for the payor, but, conversely, revenue may also be eliminated for the provider.

Improvements may be put into practice 107 following the simulation and testing described above and the application of change management principles. The specific improvements to implement the new value stream depend on the results, and may include, for example, elimination or addition of specific tests, refinement of “mistake-proofed” forms with forcing functions embedding evidence-based practices to ensure that tests are used consistently with the refined evidence-based value stream, changes in patient flow, and the like.

The implementation of improvements may require changes in standard order sets, use of algorithms, creation of forms, online test ordering forms with decision rules built-in, and the like. Staff and physicians may require additional training to learn and implement the new standard, waste-free flow of a patient through one episode. Printed materials may be created to facilitate this training; for example, new standard work documents and new procedures for payor and provider, new printed materials for patients to educate them on the new flow and any impact upon them, scripts for staff to use in appointment scheduling, and the like. The flow of the patient may become smoother and faster as non-value-added cycles are removed from the process. The flow of the providers and support staff may improve as waste is removed from their processes. As a result, the payor may be paying the provider less per episode of care.

Following implementation, the improved system may be continually reviewed and/or improved by beginning once again with gathering data 101, and proceeding through the inventive process.

### EXAMPLE

The following example is provided to better illustrate the claimed invention and is not to be interpreted as limiting the scope of the invention. To the extent that specific materials or processes are mentioned, it is merely for purposes of illustration and is not intended to limit the invention. One skilled in the art may develop equivalent components without the exercise of inventive capacity and without departing from the scope of the invention.

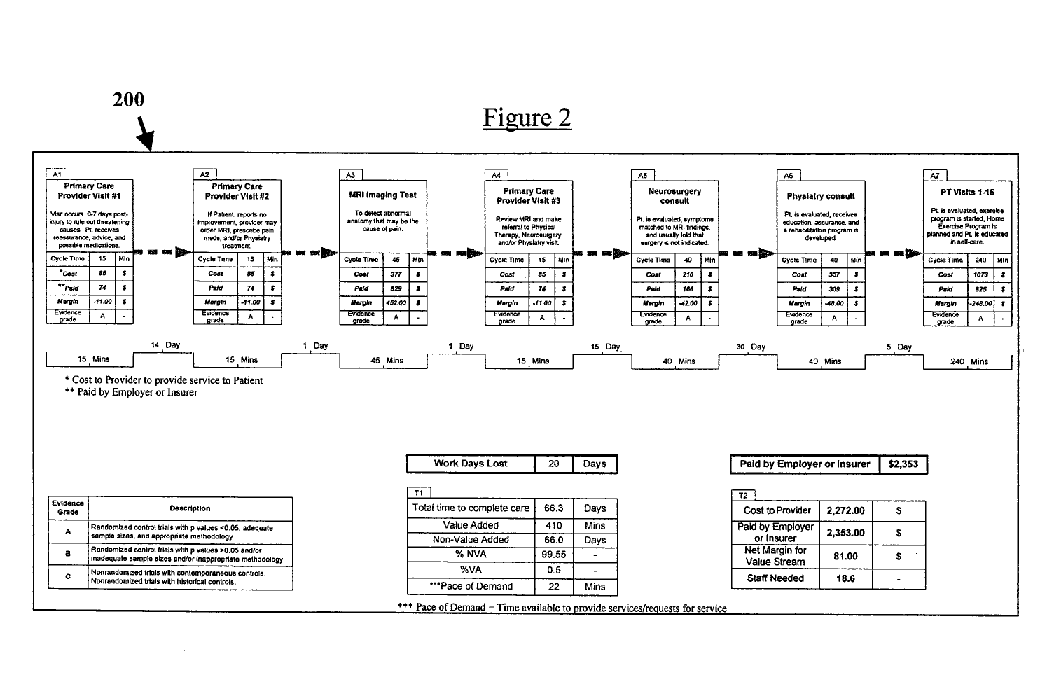

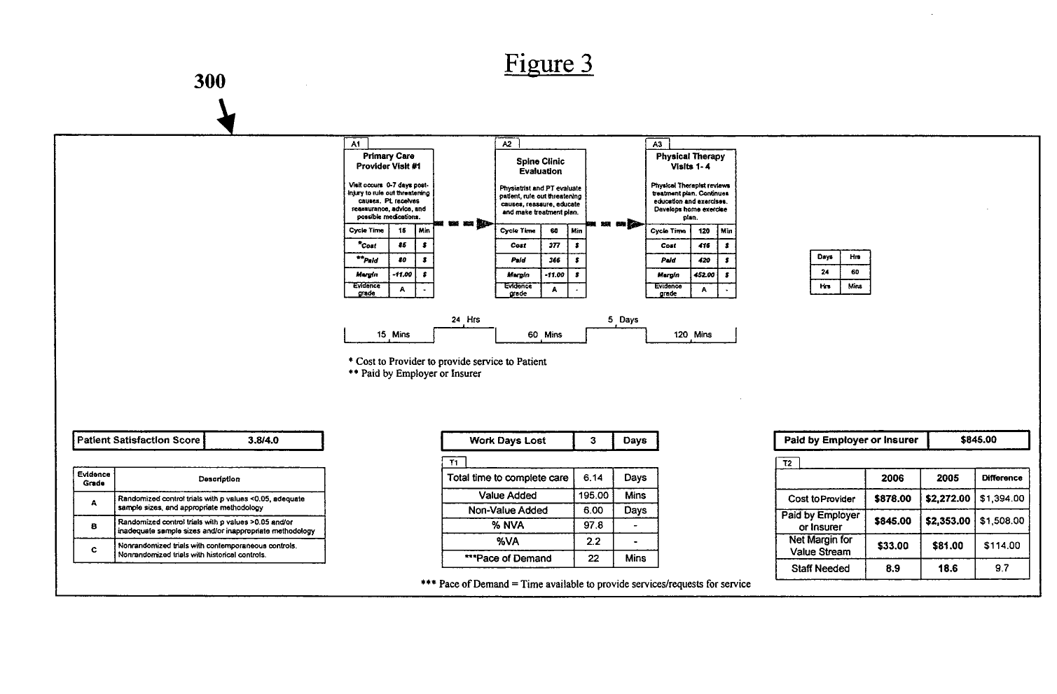

The inventor implemented the methods of the present invention to develop a future state value stream map for a spine clinic; specifically, for the treatment of low back pain. In the current state value stream map 200 (FIG. 2), the value stream showed a lead time of 66.3 days, while in the future value stream 300 (FIG. 3), the lead time was reduced to 6.14 days. The changes implemented included elimination of a magnetic resonance imaging (“MRI”) test (FIG. 2, No. A3), elimination of several physician visits (FIG. 2, A2, A4), and patient care through a physiatrist/physical therapist team in a spine clinic (FIG. 3, A2). The costs of treatment of an episode of lower back pain were reduced from $2,272.00 (in the current state value stream map; FIG. 1) to $878.00 (in the future state value stream map; FIG. 2). The number of work days lost was reduced from 20 to 3.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein

What is claimed is:

1. A method for delivery of a healthcare service, comprising:
gathering data relating to the delivery of said healthcare service;
manipulating said data to place it in a format suitable for a current state value stream map;
creating said current state value stream map;
identifying waste in said current state value stream map;
creating a future state value stream map including at least one proposed change, by removing at least a portion of said waste; and
implementing said healthcare service based on said future state value stream map.

2. The method of claim 1, further comprising testing and/or simulating said at least one proposed change prior to implementing said healthcare service based on said future state value stream map.

3. The method of claim 1, wherein said current state value stream map comprises a current map item selected from the group consisting of cycles of work, wait times between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, patient satisfaction scores associated with the current state value stream, work days lost associated with the current state value stream, and combinations thereof.

4. The method of claim 1, wherein creating said current state value stream map further comprises creating said current state value stream map for one episode treatment group.

5. The method of claim 1, wherein creating a future state value stream map including at least one proposed change further comprises utilizing an evidence-based medicine approach to propose said proposed change.

6. The method of claim 1, wherein said waste is selected from the group consisting of a defect, an overproduction, a transportation, a waiting, an inventory, a motion, a processing, an inefficiency, a non-value added element of said current state value stream map, a lengthy delay between cycles, an expensive treatment and/or tests determined to be redundant and/or that adds little or no value to the final product, a provider visit that is unnecessary, and combinations thereof.

7. The method of claim 1, wherein said future state value stream map depicts a provision of said healthcare service with substantially all of said waste having been eliminated.

8. The method of claim 1, wherein said future state value stream map comprises a future map item selected from the group consisting of a cycle of work, a wait time between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, a new measure that results from the implementation of said future state value stream map, a reduced lead time, a reduced number of days of work lost, an increase in patient satisfaction metrics, an increased percentage of value-added time in said lead time, a non-value-added activity eliminated, and combinations thereof.

9. The method of claim 1, wherein said creating said future state value stream map further comprises conducting a negotiation between a payor and a provider or among the payor, the provider and an employer.

10. The method of claim 1, wherein said implementing said healthcare service based on said future state value stream map further comprises an implementation activity selected from the group consisting of changing a standard order set, using an algorithm, creating a form, utilizing online test ordering forms with decision rules built-in, training staff and/or physicians to learn and implement said healthcare service based on said future state value stream map, creating printed material to facilitate said training of staff and/or physicians, and combinations thereof.

11. The method of claim 1, wherein a payor pays a provider less per episode of care as a result of said implementing said healthcare service based on said future state value stream map.

12. The method of claim 1, wherein a payor pays a provider more per episode of care as a result of said implementing said healthcare service based on said future state value stream map and an outcome is of greater quality and/or a patient satisfaction is higher and/or a number of work days lost is lower.

13. The method of claim 1, further comprising repeating said method to further improve said delivery of said healthcare services.

14. A future state value stream map for the delivery of a healthcare service, produced by the process of:
gathering data relating to the delivery of said healthcare service;
manipulating said data to place it in a format suitable for a current state value stream map;
creating said current state value stream map;
identifying waste in said current state value stream map; and
creating said future state value stream map including at least one proposed change, by removing at least a portion of said waste.

15. The future state value stream map of claim 14, wherein said process further comprises utilizing an evidence-based medicine approach to propose said proposed change.

16. The future state value stream map of claim 14, wherein said waste is selected from the group consisting of a defect, an overproduction, a transportation, a waiting, an inventory, a motion, a processing, an inefficiency, a non-value added element of said current state value stream map, a lengthy delay between cycles, an expensive treatment and/or tests determined to be redundant and/or that adds little or no value to the final product, a provider visit that is unnecessary, and combinations thereof.

17. The future state value stream map of claim 14, wherein said future state value stream map depicts a provision of said healthcare service with substantially all of said waste having been eliminated.

18. The future state value stream map of claim 14, further comprising a future map item selected from the group consisting of a cycle of work, a wait time between cycles of work, a work in process between cycles, an information and/or material flow, a lead time, a cycle time, a percentage of time that is value-added, a percentage of time that is non-value-added, a total of cycle times, a comparison of provider costs with payor reimbursement for a service, a new measure that results from the implementation of said future state value stream map, a reduced lead time, an increased percentage of value-added time in said lead time, a non-value-added activity eliminated, and combinations thereof.

19. The future state value stream map of claim 14, wherein said process further comprises conducting a negotiation between a payor and a provider and/or a negotiation among the payor, the provider and an employer.
